(19) United States
(12) Patent Application Publication
(10) Pub. No.: US 2013/0288352 A1
(43) Pub. Date: Oct. 31, 2013

(54) Bioindicator

(71) Applicants: Thomas Jehle, Rheinfelden (DE); Daniel Scherzinger, Zurich (CH); Volker Sigwarth, Sisseln (CH)

(72) Inventors: Thomas Jehle, Rheinfelden (DE); Daniel Scherzinger, Zurich (CH); Volker Sigwarth, Sisseln (CH)

(73) Assignee: SKAN AG, Allschwil (CH)

(21) Appl. No.: 13/978,368

(22) PCT Filed: Jan. 5, 2012
(86) PCT No.: PCT/CH2012/000002

§ 371 Date: Jul. 3, 2013

(30) Foreign Application Priority Data
Jan. 5, 2011 (CH) 20/11

(51) Int. Cl. C12Q 1/22

(52) U.S. Cl. CPC C12Q 1/22
USPC 435/287.4

(57) ABSTRACT

The bioindicator (1) intended for verifying the effectiveness of a sterilization process carried out in a working chamber (90) of a containment system initially has an outer microporous envelope (3) which is permeable to a sterilizing agent (8) but impermeable to microorganisms (20). The carrier (2) accommodated in the interior (10) of the bioindicator (1) is provided with a determined quantity and type of microorganisms (20). The envelope (3) comprises a fixing means (4) which is intended for temporarily and detachably positioning the bioindicator (1) in the working chamber (90). The fixing means (4) is an adhesive strip which is provided with a detachable coating (40). The carrier (2) may be detachably fixed in the interior of the envelope (3) by means of a receptacle (30). The receptacle (30) takes the form of a pocket, an adhesive joint or tear-off point. On the outer surface (34), the envelope (3) has a labeling area (33) to which a symbol carrier (5) may be affixed permanently or detachably. The labeling area (33) directly and/or the symbol carrier (5) are intended for application of a code (50). The interior (10) is defined by a primary closure (32) which may be supplemented with a repeatedly toggleable secondary closure (35).

### FIELD OF THE INVENTION

The present invention relates to a bioindicator for verifying the effectiveness of a sterilization process carried out in the working chamber of a containment system. The bioindicator has a microporous envelope on the outside which is permeable to a sterilizing agent from the outside into the interior of the envelope and is impermeable to microorganisms. The envelope accommodates a carrier with a determined quantity and type of microorganisms applied thereto.

### PRIOR ART

DE 1 492 498 discloses a disinfection indicator package which comprises a semitransparent envelope of a dialyzing filter material, preferably of plastic material, which allows the disinfecting medium to pass through but retains bacteria. In the envelope is located a carrier on which a defined quantity of bacteria is applied. A nutrient medium and a color indicator are also enclosed in the envelope. The package is introduced into a disinfection chamber and the disinfection process is carried out. The package is then removed and immersed in chlorine-free water, which reaches the nutrient medium through the envelope. If disinfection was not successful, the bacteria react with the nutrient medium, this being indicated by the color indicator. No change to the color indicator, on the other hand, means that the disinfection process was successful.

WO 96/06184 describes a package as bioindicator with microorganisms applied on a carrier which is enclosed by a hydrophilic microporous envelope which allows disinfecting medium, but not the microorganisms, to pass through. Once the disinfection process has been carried out, the carrier is exposed to a nutrient medium, such that an absence of growth proves that disinfection has occurred. The nutrient medium can penetrate through the envelope and the measurement proceeds under protected laboratory conditions or the envelope is opened in a safety container and exposed therein to the nutrient medium.

The bioindicator disclosed in WO 95/06134 likewise relates to a package with an envelope which contains a carrier on which bacteria are applied. The envelope has a microporous area which allows a sterilizing agent to pass through from the outside to the inside, but does not allow bacteria to get out. The microporous area may extend over the entire envelope or only over a subregion, wherein the remaining area is covered by other materials, for example polyethylene or polypropylene. The envelope may be provided with seams, joints and seals or comprise peelable adhesive bonds.

DE 100 46 427 A1 discloses a bioindicator which is used for validating an ethylene oxide sterilization process in the medical sector even in poorly accessible areas in order to verify successful inactivation of microorganisms over an entire test specimen. Since a bioindicator pressed against the test specimen exhibits a false measurement result, an interlayer which is impermeable to ethylene oxide is provided between the bioindicator and the test specimen.

EP 0 093 920 A1 relates to a bioindicator for microbiological monitoring of gas sterilization methods. The bioindicator has a spore carrier located in a container. The container is a thermoformed package and is sealed with a film permeable to gas and water vapor through which the sterilizing agent can reach the spore carrier. For the purposes of verifying the effectiveness of the sterilization process which has been carried out, the film can be peeled off and the spore carrier then placed in an ampoule with nutrient medium. Alternatively, the container is connected to the ampoule, wherein there is a partition between the two, through which the spore carrier previously exposed to the sterilizing agent can be pushed from the container into the ampoule.

### OBJECT OF THE INVENTION

In relation to the known prior art, the object of the invention is to propose an improved bioindicator which, when used in a containment system and the subsequent laboratory test, enables advantageous practical application and reduces the complexity hitherto associated therewith. Cost-effective mass production of the bioindicator should moreover be possible.

### SUMMARY OF THE INVENTION

The bioindicator is intended for verifying the effectiveness of a sterilization process carried out in a working chamber of a containment system. The bioindicator has a microporous envelope on the outside which is permeable to a sterilizing agent and impermeable to microorganisms. The outer surface is present on the envelope. A carrier with a determined quantity and type of microorganisms applied thereto is arranged in the interior of the bioindicator. The envelope has on the outer surface a fixing means which is intended for temporarily and detachably positioning the bioindicator in the working chamber.

Specific embodiments of the invention are defined below: the fixing means is an adhesive strip which is provided with a detachable coating. The carrier is detachably fixed in the interior of the envelope by means of a receptacle. The receptacle takes the form of a pocket, an adhesive joint or tear-off point.

The envelope has a labeling area on the outer surface. A symbol carrier may be affixed permanently or detachably to the labeling area. The labeling area directly and/or the symbol carrier are intended for application of a code. The symbol carrier is a sticker with a code which has been pre-produced or may be applied. A plurality of symbol carriers is provided to form a set. The symbol carriers are provided with a system of codes, the purpose of which is to permit clear assignment of the placement of the individual bioindicator in the containment system. The system of codes is made up, for example, of numeric or alphanumeric characters or as a combination of colors.

The envelope is bag-shaped or has the form of a flat sachet, in which latter case it consists of a front sheet and a congruently assembled rear sheet joined thereto. The envelope has an entrance with a primary closure to be opened for inserting and/or removing the carrier. The primary closure is used up after one opening operation or may be repeatedly toggled between closed and open states. The primary closure takes the form of a weld seam or adhesive bond, wherein the weld seam or adhesive bond extends in the manner of a seam in the peripheral zones in the mid region over the front and rear sheets and so defines the interior. From the primary closure, which takes the form of a weld seam or adhesive bond, there extend in a U-shape two webs which are spaced apart from one another and form the receptacle in the form of a pocket, into which may be inserted one end of the strip-shaped carrier.

The envelope consists of a tear-resistant, hydrophobic material which is chemically resistant to the sterilizing agent. The envelope has the same permeability to the sterilizing agent on the front and rear or with regard to the front and rear sheets. The envelope consists of acrylic copolymer, polyether sulfone (PES), polyvinylidene fluoride (PVDF), polytetrafluoroethylene (PTFE/Teflon) or polypropylene (PP).

In addition to the primary closure, the envelope may additionally be provided with a repeatedly toggleable secondary closure, which likewise defines the interior. In order to simplify handling on opening the entrance, the mutually adjacent ends of the front and rear sheets take the form of tab portions which may simply be spread apart from one another. The secondary closure consists of magnetic strips, one affixed onto the outer surfaces of each of the two sheets.

The magnetic strips are affixed in principle and for the most part congruently with the course of the primary closure. The magnetic strips are U-shaped precut parts and in each case have a central web and two free legs. The central webs of the magnetic strips are located at a transition from the tab portions to the adjacent portion of the primary closure, while the two free legs of the magnetic strips in each case extend in the longitudinal direction relative to the receptacle.

### BRIEF DESCRIPTION OF THE ATTACHED DRAWINGS

In the drawings:

FIG. 1A—shows a side view of a bioindicator according to the invention in a first version, with the carrier brought close to the envelope, in the open state;

FIG. 1B—shows a side view of the structure according to FIG. 1A, with the carrier inserted through the entrance of the envelope, in the open state;

FIG. 1C—shows a side view of the structure according to FIG. 1B, with the carrier fixed to the receptacle in the envelope, in the open state;

FIG. 1D—shows a side view of the structure according to FIG. 1C, in the closed state with symbol carrier applied to the bioindicator;

FIG. 1E—shows a side view of the structure according to FIG. 1D, with the bioindicator introduced into the working chamber of the containment system, in the closed state, and the coating already detached from the fixing means;

FIG. 1F—shows a side view of the structure according to FIG. 1E, in the closed state, with the fixing means detachably positioned on the wall of the working chamber;

FIG. 2A—shows a perspective view of a bioindicator in a second version, in the closed state;

FIG. 2B—shows a different perspective view of the structure according to FIG. 2A;

FIG. 2C—shows a perspective view of the structure according to FIG. 2A, in the open state;

FIG. 3A—shows a perspective view of the bioindicator according to FIG. 2A, with an additional secondary closure, in the closed state;

FIG. 3B—shows a different perspective view of the structure according to FIG. 3A; and

FIG. 3C—shows a perspective view of the structure according to FIG. 3A, in the open state.

### EXEMPLARY EMBODIMENT

A detailed description is provided below with reference to the attached drawings of the bioindicator according to the invention in the first version thereof with the sequence of introducing the carrier into the envelope and affixing the bioindicator in a containment system. The description of the bioindicator in the second version is limited to the open and closed state and to the optional provision of an additional secondary closure.

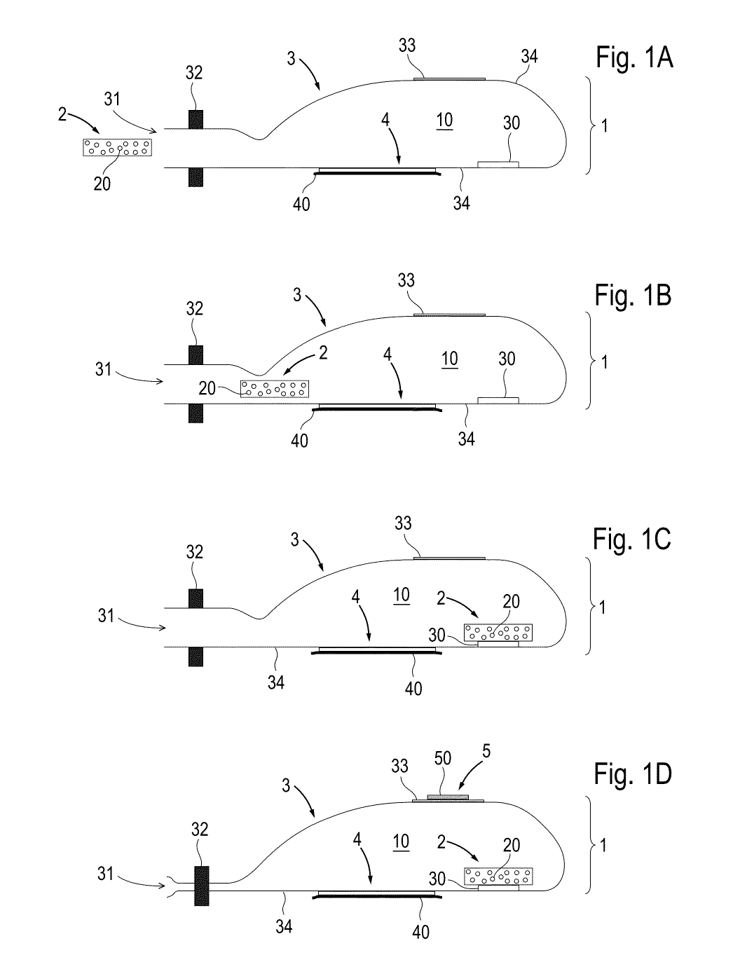

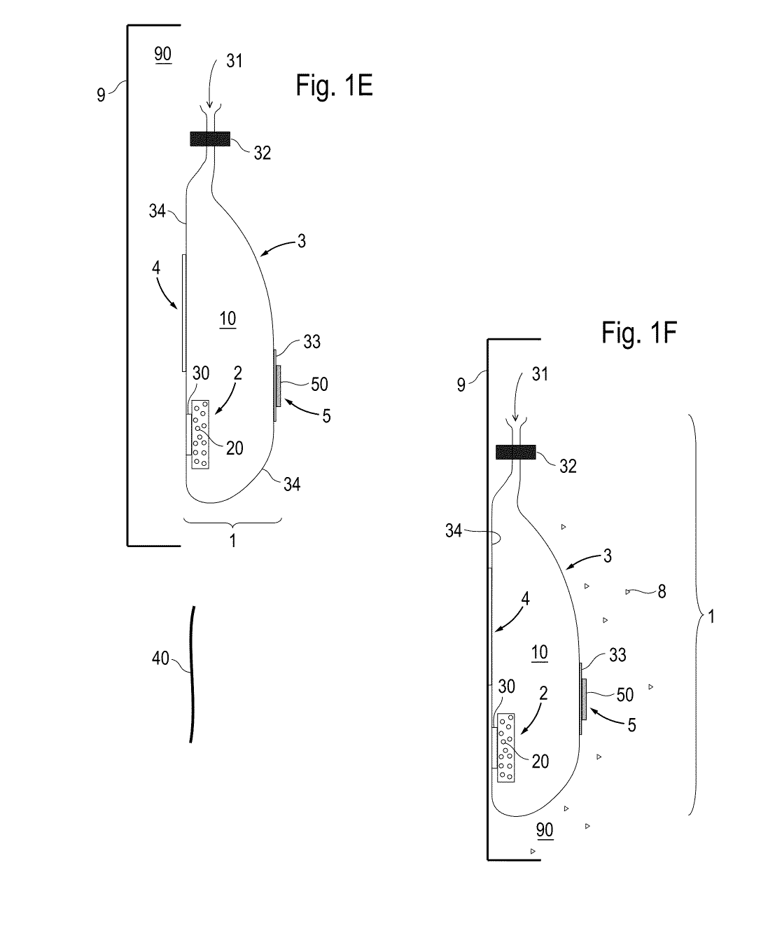

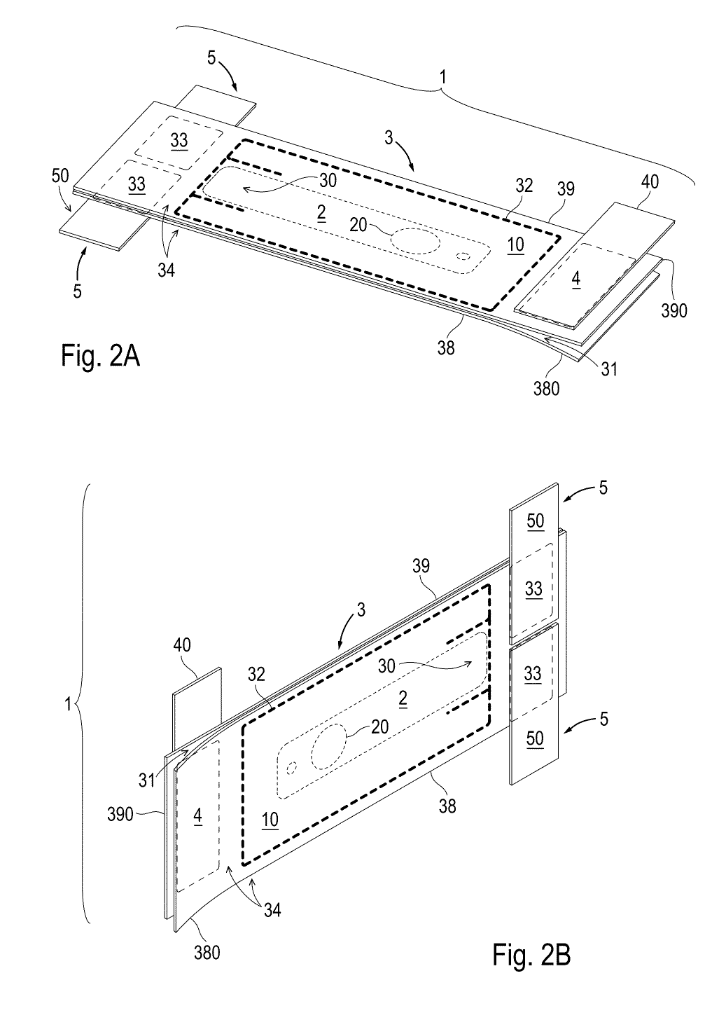

FIG. 1A

The bioindicator 1 has, in the first version thereof, an external, more or less bag-shaped microporous envelope 3, which encloses an interior 10. This interior 10 accommodates a carrier 2. Microorganisms 20 of a determined quantity and type are applied to the carrier 2. The envelope 3 is permeable to a sterilizing agent 8 (see FIG. 1F), but impermeable to microorganisms 20. The envelope 3 is tear-resistant and chemically resistant to the sterilizing agent 8. The carrier 2 is detachably fixable in the interior of the envelope 3 by means of a receptacle 30, wherein the receptacle 30 takes the form of an adhesive joint or tear-off point.

The envelope 3 has an entrance 31 for inserting and/or removing the carrier 2. The entrance 31 is provided with a primary closure 32 which is intended to convert the entrance 31 at least once from an open state into a closed state and at least once from the closed state into the open state. The primary closure 32 has self-adhesive inner sides which face towards one another. On an outer surface 34, the envelope 3 has a fixing means 4 which is intended for temporarily and detachably positioning the bioindicator 1 on the wall 9 within the working chamber 90 of the containment system (see FIG. 1F). The fixing means 4 is an adhesive strip which is provided with a repeatedly adhering and repeatedly detachable coating 40. Alternatively, the fixing means 4 may also be overlappingly affixed as an attachment to the envelope 3 or the outer edge thereof. A labeling area 33 is provided on the outside of the envelope 3 for affixing a symbol carrier 5 (see FIG. 1D). At this moment, the primary closure 32 is open and the carrier 2 is still completely outside the envelope 3, but already brought close to the primary closure 32. The carrier 2 provided with the microorganisms 20 is introduced into the envelope 3 while maintaining biosafety.

FIG. 1B

With the primary closure 32 open, the carrier 2 has been inserted through the entrance 31 of the envelope 3 into the interior thereof. The carrier 2 is not yet fixed to the receptacle 30.

FIG. 1C

The carrier 2 is detachably fixed to the receptacle 30 inside the envelope 3, and the primary closure 32 is still open.

FIG. 1D

The primary closure 32 is now closed and the entrance 31 is thus blocked. In accordance with the characteristics of the envelope 3, the microorganisms 20 located on the carrier 2 cannot get out. The symbol carrier 5 is detachably affixed to the labeling area 33. A suitable symbol carrier 5 is in particular a sticker with a preapplied code 50, wherein the user is advantageously provided with a set of such symbol carriers 5 with a system of codes 50 (for example numeric, alphanumeric or a combination of colors) and the user selects an appropriate symbol carrier 5 from the set in accordance with the intended placement of the bioindicator 1 in the containment system. Alternatively, the code 50 on the symbol carrier 5 is not pre-produced, but instead applied by the user him/herself in accordance with a user-implemented system.

It goes without saying that, independently of the symbol carrier 5 with the code 50 for clear assignment of the placement of the individual bioindicator 1 in the containment system, each bioindicator 1 is provided with the regulatorily required details regarding manufacturer/distributor, the organisms contained therein and a batch identifier.

FIG. 1E

The bioindicator 1 is placed in the working chamber 90 of a containment system by initially bringing the outer surface 34 of said bioindicator close to the intended area on the selected wall 9. The symbol carrier 5 is thus still oriented visibly for the user in the interior of the containment system. The coating 40 is detached from the fixing means 4, this advantageously proceeding prior to introduction of the bioindicator 1 into the working chamber 90.

FIG. 1F

The bioindicator 1 is now placed in the intended area on the selected wall 9 in the working chamber 90 of the containment system. The fixing means 4 present on the envelope 3 holds the bioindicator 1 detachably on the wall 9. In general, in particular depending on the size of the working chamber 90, a number of such bioindicators 1 are placed in all regions of the working chamber 90 for verification of the effectiveness of the sterilization process carried out therein. All the installed bioindicators 1 with the respective code 50 which is applied to the individual symbol carrier 5 are listed in a record indicating the specific placement thereof in the working chamber 90. It may thus be established whether sterilization was successful in all regions of the working chamber 90 or in which specific regions sterilization proceeded unsatisfactorily.

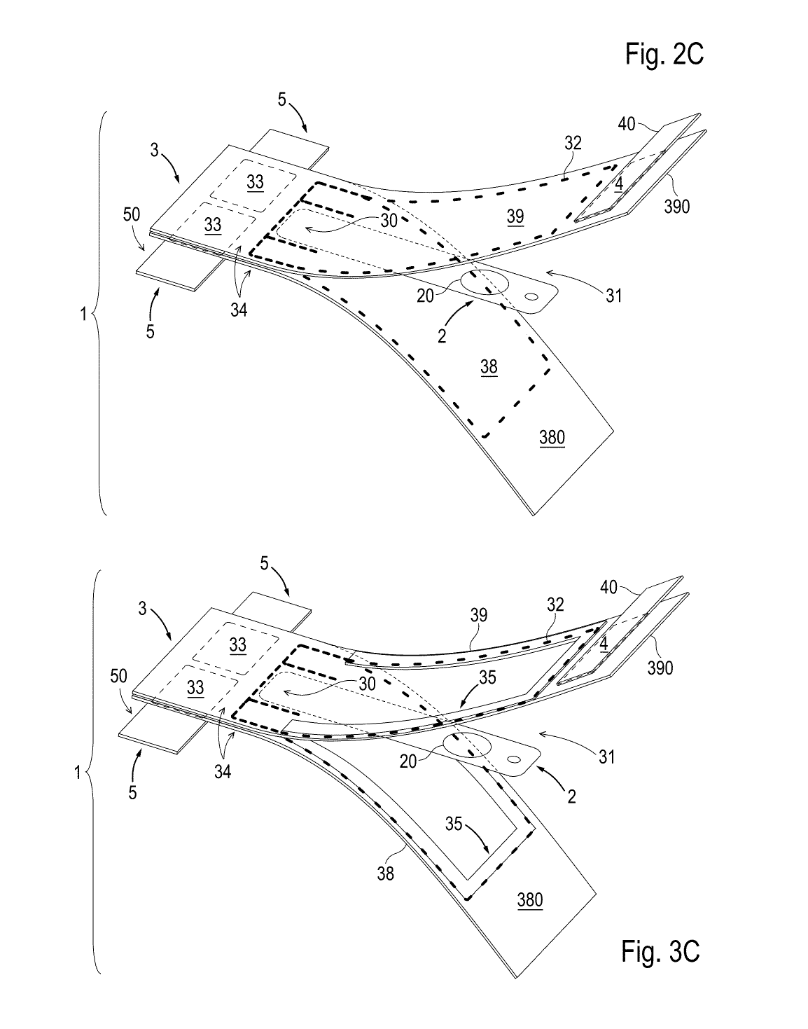

FIGS. 2A to 2C

The bioindicator 1 has, in the second version thereof, an external, flat sachet-shaped microporous envelope 3, which encloses the interior 10. The envelope 3 substantially consists of a front sheet 38 and a congruently assembled rear sheet 39 joined thereto. The front and rear sheets 38,39 are joined together in the mid region in the manner of a seam in the peripheral zones with a weld seam which forms the primary closure 32 and thus subdivides the interior 10, in which the carrier 2 is located. From the peripheral weld seam there extend in a U-shape two weld webs which are spaced apart from one another such that the pocket-shaped receptacle 30 is obtained, into which is inserted one end of the strip-like carrier 2. The carrier 2 is in turn intended for the application of microorganisms 20 (in a determined quantity and type) and, to ensure biosafety, is enclosed within the envelope 3.

The envelope 3 is permeable to the sterilizing agent 8—conventionally H₂O₂ with a concentration of for example 35% or 50% depending on the particular application—, but impermeable to microorganisms 20 and chemically resistant to the sterilizing agent 8. The envelope 3 is advantageously tear-resistant and hydrophobic, wherein the front sheet 38 and the rear sheet 39 have the same permeability to the sterilizing agent 8. Acrylic copolymer, polyether sulfone (PES), polyvinylidene fluoride (PVDF), polytetrafluoroethylene (PTFE/Teflon) or polypropylene (PP) are particularly suitable for the envelope 3.

In the state as manufactured, prior to use of the bioindicator 1 with the carrier 2 inserted inside, the primary closure 32 is closed and the entrance 31 is thus barred (see FIGS. 2A, 2B). The weld seam forming the primary closure 32 between the front and rear sheets 38,39 is advantageously still self-adhesive once torn open in order to convert the entrance 31 from the open to the closed state at least once. Adjacent one narrow side, the envelope 3 has on the outer surface 34—for example on the rear sheet 39—a fixing means 4 taking the form of an adhesive strip which is provided with the repeatedly adhering and repeatedly detachable coating 40 in order to position the bioindicator 1 temporarily and detachably on the wall 9 within the working chamber 90 of the containment system (see FIG. 1F). Adjacent the other narrow side, the envelope 3 has on the outer surface 34—for example on the front sheet 38—a labeling area 33 for affixing or applying a code 50, which may be written on a symbol carrier 5 fixable to the labeling area 33 or directly on the labeling area 33. The symbol carrier 5 could for example be a sticker redetachable from the labeling area 33 with the code 50 already applied.

Once the weld seam has been torn open, the primary closure 32 is in the open state and the entrance 31 provides access to the interior 10, and the carrier 2 may be pulled out of the receptacle 30 (see FIG. 2C). In order to simplify handling on tearing open, the ends on the front and rear sheets 38,39—more or less adjacent to the fixing means 4—take the form of unwelded tab portions 380,390 which can be simply spread apart. Depending on their rigidity, the tab portions 380,390 are already slightly bent open prior to use of the bioindicator 1, such that the user has simple access (see FIGS. 2A, 2B).

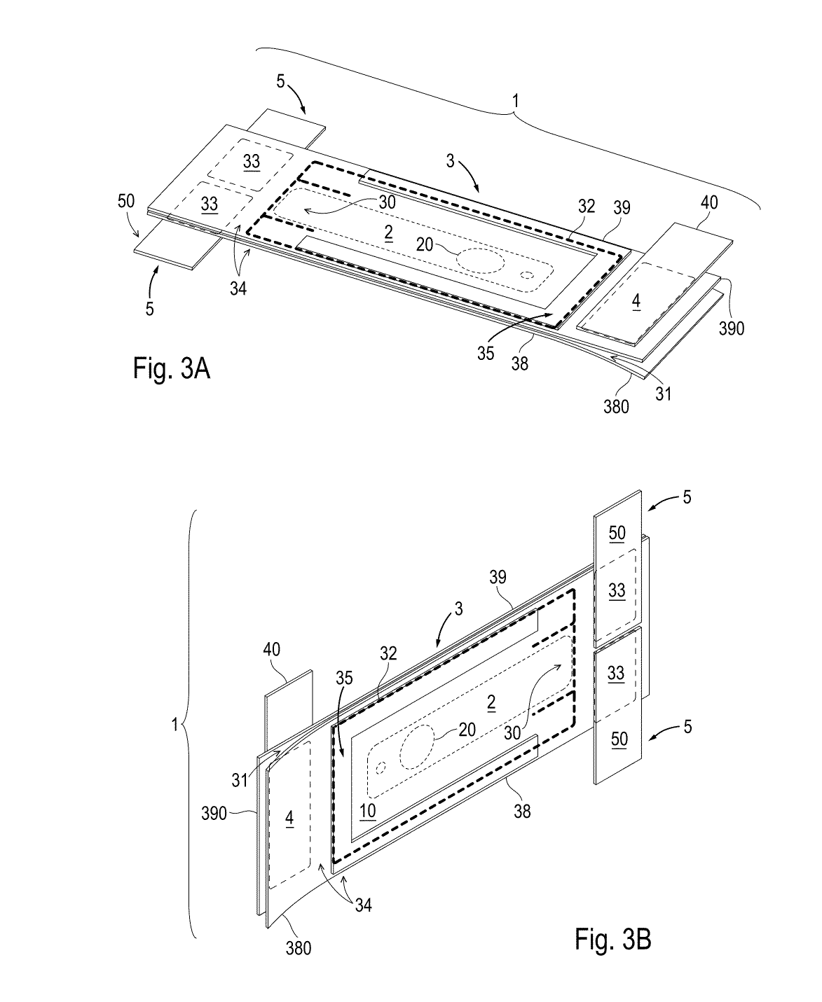

FIGS. 3A to 3C

In this sequence of figures, the bioindicator 1 according to the second version is additionally provided with a secondary closure 35, which is advantageously formed with magnetic strips in the form of U-shaped precut parts. The secondary closure 35 enables repeated opening and reclosure of the entrance 31. A magnetic strip is affixed, in principle congruently with the course of the weld seam, on the respective outer surface 34 of each of the two sheets 38,39. The central webs of the magnetic strips here come to rest at the transition from the tab portions 380,390 to the adjacent portion of the weld seam, while the in each case two free legs of the magnetic strips extend in the longitudinal direction relative to the receptacle 30. While the weld seam is still intact, the primary closure 32 is in any event closed, and, once the weld seam has been torn open, the secondary closure 35 brings the two sheets 38,39 close together and thus closes the entrance 31 (see FIGS. 3A, 3B).

Once the weld seam has been torn open and the secondary closure 35 pulled apart, the entrance 31 into the interior 10 of envelope 3 is wide open, such that the carrier 2 can be removed from the receptacle 30 (see FIG. 3C). The two U-shaped magnetic precut parts diverge from one another towards the tab portions 380,390 in the manner of a beak. If the two sheets 38,39 are brought back towards one another, the secondary closure 35 comes into effect and holds the entrance 31 closed even after the weld seam has previously been torn open.

1. A bioindicator (1) which is intended for verifying the effectiveness of a sterilization process carried out in a working chamber (90) of a containment system, wherein the bioindicator (1) comprises:
a) a microporous envelope (3) on the outside which has an outer surface (34) and is permeable to a sterilizing agent (8) but impermeable to microorganisms (20); and
b) a carrier (2) arranged in an interior (10) and with a determined quantity and type of microorganisms (20) applied thereto, characterized in that
c) the envelope (3) comprises fixing means (4) on the outer surface (34), which fixing means is intended for temporarily and detachably positioning the bioindicator (1) in the working chamber (90).

2. The bioindicator (1) as claimed in claim 1, characterized in that the fixing means (4) is an adhesive strip which is provided with a detachable coating (40).

3. The bioindicator (1) as claimed in one of claims 1 and 2, characterized in that
a) the carrier (2) is detachably fixed in the interior (10) of the envelope (3) by means of a receptacle (30); and
b) the receptacle (30) takes the form of a pocket, an adhesive joint or tear-off point.

4. The bioindicator (1) as claimed in one of claims 1 to 3, characterized in that
a) the envelope (3) has a labeling area (33) on the outer surface (34);
b) a symbol carrier (5) may be affixed permanently or detachably to the labeling area (33); and
c) the labeling area (33) directly and/or the symbol carrier (5) is/are intended for application of a code (50).

5. The bioindicator (1) as claimed in claim 4, characterized in that the symbol carrier (5) is a sticker with a code (50) which has been pre-produced or may be applied.

6. The bioindicator (1) as claimed in one of claims 4 and 5, characterized in that
a) a plurality of symbol carriers (5) is provided to form a set;
b) the symbol carriers (5) are provided with a system of codes (50), the purpose of which is to permit clear assignment of the placement of the individual bioindicator (1) in the containment system; and
c) the system of codes (50) is made up, for example, of numeric or alphanumeric characters or as a combination of colors.

7. The bioindicator (1) as claimed in one of claims 1 to 6, characterized in that
a) the envelope (3) is bag-shaped; or
b) the envelope (3) has the form of a flat sachet and consists of a front sheet (38) and a congruently assembled rear sheet (39) joined thereto;
c) the envelope (3) has an entrance (31) with a primary closure (32) to be opened for inserting and/or removing the carrier (2); and
d) the primary closure (32) is used up after one opening operation or may be repeatedly toggled between closed and open states.

8. The bioindicator (1) as claimed in claim 7, characterized in that
a) the primary closure (32) takes the form of a weld seam or adhesive bond; wherein
b) the weld seam or adhesive bond extends in the manner of a seam in the peripheral zones in the mid region over the front and rear sheets (38,39) and so defines the interior (10).

9. The bioindicator (1) as claimed in claim 8, characterized in that, from the primary closure (32), which takes the form of a weld seam or adhesive bond, there extend in a U-shape two webs which are spaced apart from one another and form the receptacle (30) in the form of a pocket, into which may be inserted one end of the strip-shaped carrier (2).

10. The bioindicator (1) as claimed in one of claims 1 to 9, characterized in that the envelope (3):
a) consists of a tear-resistant, hydrophobic material which is chemically resistant to the sterilizing agent (8); and
b) on the front and rear or with regard to the front and rear sheets (38,39) has the same permeability to the sterilizing agent (8).

11. The bioindicator (1) as claimed in one of claims 1 to 10, characterized in that the envelope (3) consists of acrylic copolymer, polyether sulfone (PES), polyvinylidene fluoride (PVDF), polytetrafluoroethylene (PTFE/Teflon) or polypropylene (PP).

12. The bioindicator (1) as claimed in one of claims 1 to 11, characterized in that, in addition to the primary closure (32), the envelope (3) is provided with a repeatedly toggleable secondary closure (35) which likewise defines the interior (10).

13. The bioindicator (1) as claimed in one of claims 7 to 12, characterized in that, in order to simplify handling on opening the entrance (31), the mutually adjacent ends of the front and rear sheets (38,39) take the form of tab portions (380,390) which can be simply spread apart from one another.

14. The bioindicator (1) as claimed in one of claims 12 and 13, characterized in that the secondary closure (35) consists of magnetic strips, one affixed onto the outer surfaces (34) of each of the two sheets (38,39).

15. The bioindicator (1) as claimed in claim 14, characterized in that
a) the magnetic strips are affixed in principle and for the most part congruently with the course of the primary closure (32);
b) the magnetic strips are U-shaped precut parts and in each case have a central web and two free legs; wherein
c) the central webs of the magnetic strips come to rest at a transition from the tab portions (380,390) to the adjacent portion of the primary closure (32); and
d) the in each case two free legs of the magnetic strips extend in the longitudinal direction relative to the receptacle (30).
